(19) United States
(12) Patent Application Publication
(10) Pub. No.: US 2023/0148975 A1
(43) Pub. Date: May 18, 2023

(54) SHROUDED X-RAY DEVICE

(71) Applicant: LUMAFIELD, Cambridge, MA (US)

(72) Inventors: Adam DAMIANO, Somerville, MA (US); Brian CHAN, Cambridge, MA (US)

(21) Appl. No.: 17/987,705

(22) Filed: Nov. 15, 2022

Related U.S. Application Data
(60) Provisional application No. 63/280,064, filed on Nov. 16, 2021.

(51) Int. Cl. A61B 6/03; A61B 6/08; A61B 6/00

(52) U.S. Cl. CPC A61B 6/032; A61B 6/08; A61B 6/4085

(57) ABSTRACT

Systems, methods, apparatuses, and computer program products for the detection of X-ray electromagnetic radiation using a scintillator are provided. In one example embodiment, at least one X-ray device may comprise an X-ray source configured to emit an X-ray cone, a scintillator, a detector, and at least one shroud positioned to block stray light from reaching the detector.

### CROSS REFERENCE TO RELATED APPLICATION

This application claims the benefit of, and priority to, U.S. Provisional Pat. Application No. 63/280,064, filed on Nov. 16, 2021. The entire content of the above-referenced application is hereby incorporated by reference.

### TECHNICAL FIELD

Some example embodiments may generally relate to the detection of X-ray electromagnetic radiation using a scintillator and/or a shroud. For example, certain example embodiments may relate to systems and/or methods for nonintrusive scanning of objects using X-ray electromagnetic radiation.

### BACKGROUND

X-ray devices, such as computed tomography (CT) devices, may be used to detect defects and/or damage in an object without disassembling the object. However, current X-ray detection equipment needs improvement because they are cost-prohibitive for certain analyses, too large or bulky to be used in certain situations, unable to form images of an object’s interior with the appropriate resolution, and other problems known in the field. Set forth herein are solutions to these and other problems known in the field.

### SUMMARY

In accordance with various example embodiments, an X-ray device may include at least one X-ray source configured to emit an X-ray cone, a scintillator, a detector, and at least one shroud positioned to block stray light from reaching the detector.

In certain example embodiments, the shroud may block between 90% to 99.999% of stray light from reaching the detector.

In some example embodiments, the shroud may block at least 80% of stray light from reaching the detector.

In various example embodiments, the shroud may block at least 90% of stray light from reaching the detector.

In certain example embodiments, the shroud may block at least 95% of stray light from reaching the detector.

In some example embodiments, the shroud may block at least 99% of stray light from reaching the detector.

In various example embodiments, the shroud may block stray light to less than 0.1% of a signal.

In certain example embodiments, the shroud may block stray light at or near a read-noise of the detector.

In some example embodiments, the shroud may block incident light on the detector to only scintillated light from the scintillator.

In various example embodiments, the scintillator may be an organic scintillator, an inorganic scintillator, an organic-inorganic scintillator, or any combination thereof.

In various example embodiments, the scintillator may be an organic scintillator material, an inorganic scintillator material, an organic-inorganic scintillator material, or any combination thereof.

In certain example embodiments, the scintillator may be an inorganic scintillator selected from alkali metal halides optionally comprising dopants, phosphors, quantum dots, and combinations thereof.

In some example embodiments, the scintillator may include at least one of gadolinium oxysulfide (Gadox), terbium-activated Gadox, cesium iodide, or a combination thereof.

In various example embodiments, the scintillator form factor may be a thin film.

In certain example embodiments, the detector may include an optical camera, a charge-coupled device detector, a photodiode, or any combination thereof.

In some example embodiments, the optical camera may include a complementary metal-oxide-semiconductor digital camera sensor.

In various example embodiments, the optical camera may include a red-green-green-blue Bayer filter.

In certain example embodiments, the optical camera may include a monochromatic optical camera.

In some example embodiments, the optical camera may include a back-side-illuminated sensor.

In various example embodiments, the optical camera may include a front-side-illuminated sensor.

In certain example embodiments, the shroud may be positioned between the at least one X-ray source and the detector.

In some example embodiments, the shroud may be flat and have an opening allowing at least 90% of the X-ray light cone to transmit through the shroud.

In various example embodiments, the shroud may include a tapered and truncated tip matching an optical inlet of the detector.

In certain example embodiments, the X-ray light cone may include a right-circular cone comprising an angle between 20°-90°.

In some example embodiments, a long-axis of the scintillator and a long-axis of the shroud may be parallel.

In various example embodiments, the scintillator may have a length and a width, the shroud may have a length and a width, the length of the scintillator may be parallel to the length of the shroud, and the width of the scintillator may be parallel to the width of the shroud.

In certain example embodiments, further comprising one or more mirrors configured to reflect light from the scintillator to the detector.

In some example embodiments, the one or more mirrors may be configured to reflect light 90° from the scintillator to the detector.

In various example embodiments, the one or more mirrors may be configured to reflect light 45° from the scintillator to the detector.

In certain example embodiments, a first mirror of the one or more mirrors may be mounted 45° with respect to the scintillator, and a second mirror of the plurality of mirrors may be mounted 90° with respect to the first mirror.

In some example embodiments, the one or more of mirrors may be configured to reflect light 180°.

In various example embodiments, the first mirror may be positioned approximately 200 millimeters from the scintillator, the second mirror may be positioned approximately 300 millimeters from the first mirror, and the detector may be positioned approximately 300 millimeters from the second mirror.

In certain example embodiments, the detector may be positioned between the shroud and the at least one X-ray source.

In some example embodiments, the scintillator may include a panel having a length of 320 millimeters and a width of 320 millimeters.

In various example embodiments, the scintillator may include a plurality of mounting tabs positioned within 20 millimeters of an edge of the panel.

In certain example embodiments, the shroud may include a first component and a second component, and the first component may be longer than the second component.

In some example embodiments, the first component and the second component may form an angle less than 180°.

In various example embodiments, the first component and the second component may form an angle of about 40°-60°.

In certain example embodiments, the first component and the second component may form an angle of about 45°.

In some example embodiments, the shroud may be transparent to X-ray light.

In various example embodiments, at least 80% of the X-ray light cone may transmit through the shroud.

In certain example embodiments, at least 90% of the X-ray light cone may transmit through the shroud.

In some example embodiments, the scintillator and the shroud may not be parallel.

In various example embodiments, the shroud may be at least 50% opaque to wavelengths of light detectable by the detector.

In certain example embodiments, the shroud may be 100% opaque to wavelengths of light detectable by the detector.

In some example embodiments, light detectable by the detector may include infrared and visible light.

In various example embodiments, the X-ray light cone may include X-rays selected from soft X-rays and hard X-rays.

In certain example embodiments, the X-ray light may be generated by an electrode having a voltage in the range of 20 kV - 225 kV.

In some example embodiments, the X-ray light may be in the range of 5 picometers - 60 picometers.

In various example embodiments, the stray light may be selected from light from the at least one X-ray source, light from at least one X-ray source controller, light from limit switches, light from the detector, and reflected light.

In certain example embodiments, the stray light may be non-scintillated light.

In some example embodiments, the shroud may include fabric, foam, sheet metal, paper, cardboard, or any combination thereof.

In various example embodiments, the X-ray device may not comprise one or more mirrors to reflect light from the scintillator to the detector.

In certain example embodiments, the X-ray device may be an X-ray computed tomography device.

In some example embodiments, the X-ray device may further include a motion system configured to reposition the X-ray device during a scan.

In various example embodiments, at least one of the at least one X-ray source, the detector, and a scan target may be configured to be moved by the motion system during a scan.

In certain example embodiments, the at least one X-ray source may be configured to emit an X-ray cone as a pencil beam, fan beam, or cone beam.

In some example embodiments, the stray light may include at least one of visible light, infrared light, or ultraviolet light.

In accordance with various example embodiments, a method of providing a radiograph may include detecting optical or infrared scintillated light using the X-ray device.

In accordance with some example embodiments, a method may include at least one X-ray source transmitting X-rays towards a scintillator. The X-rays may pass through an object to be imaged. In response to receiving the X-rays, the scintillator may emit visible light.

In certain example embodiments, the method may further include moving, by a motion system, at least one of the at least one X-ray source, the detector, and a scan target.

In some example embodiments, the stray light may include at least one of visible light, infrared light, or ultraviolet light.

A detector may detect the visible light from the scintillator. A shroud may be positioned to block the detector from at least one of stray light or X-ray light from an X-ray source.

In some embodiments, the disclosure herein is useful for imaging objects using X-ray CT in environments which include stray light. The disclosure herein provides for the ability to locate X-ray CT scanners in places where they might not presently be used because of stray light challenges.

In some embodiments, the disclosure herein sets forth a method of providing at least one or more radiographs using the disclosure herein.

In some embodiments, the disclosure herein sets forth a method of providing at least one or more radiographs using an X-ray device that includes at least one X-ray source configured to emit an X-ray cone, a scintillator, a detector, and at least one shroud positioned to block stray light from reaching the detector.

### BRIEF DESCRIPTION OF THE DRAWINGS

For proper understanding of example embodiments, reference should be made to the accompanying drawings, wherein:

FIG. 1 illustrates an example of an X-ray device according to certain example embodiments.

FIG. 2 illustrates an example of another X-ray device according to various example embodiments.

FIG. 3 illustrates an example of another X-ray device according to some example embodiments.

FIG. 4 illustrates an example of a flow diagram of a method performed by an X-ray device according to certain example embodiments.

### DETAILED DESCRIPTION

It will be readily understood that the components of certain example embodiments, as generally described and illustrated in the figures herein, may be arranged and designed in a wide variety of different configurations. Thus, the following detailed description of some example embodiments of systems, methods, and apparatuses for detecting X-ray images is not intended to limit the scope of certain example embodiments, but is instead representative of selected example embodiments.

### Definitions

As used herein, the term “approximately” means any of substantially, generally, about, or similar.

As used herein, the term “back-side-illuminated sensor” means a digital image sensor with a particular arrangement of imaging elements. For example, a back-side-illuminated sensor may have (described from the top to the bottom) a lens (e.g., microlenses; top layer), a filter (e.g., a color filter), a photodiode substrate, and metal wiring (e.g., bottom layer).

As used herein, the term “CCD” means charge-coupled device.

As used herein, the term “CMOS” means complementary metal-oxide-semiconductor.

As used herein, the term “CPU” means central processing unit.

As used herein, the term “CsI” means cesium iodide.

As used herein, the term “CT” means computed tomography.

As used herein, the term “detector” means an apparatus or device configured to detect visible, ultraviolet, or infrared light.

As used herein, the term “front-side-illuminated sensor” means a digital image sensor with a particular arrangement of imaging elements. For example, a front-side-illuminated sensor may have (going from the top to the bottom) a lens (or microlenses; top layer), a filter (e.g., a color filter), metal wiring, and a photodiode substrate (bottom layer).

As used herein, the term “Gadox” means gadolinium oxysulfide.

As used herein, the term “kV” means kilovolt.

As used herein, the term “near a read-noise of the detector” means an inherent noise, or background signal, associated with the detector when in operation.

As used herein, the term “RGGB” means red-green-green-blue.

As used herein, the term “PS” means polystyrene.

As used herein, the term “PVT” means polyvinyltoluene.

As used herein, the term “scintillator” means a material which emits visible, ultraviolet, and/or infrared light when excited by X-ray radiation.

As used herein, the term “shroud” means a covering that blocks visible, ultraviolet, and/or infrared light from reaching a detector.

As used herein, the term “signal” means an image pixel intensity value, which corresponds to the intensity of the light incident on the sensor pixel.

As used herein, the term “stray light” means visible light, infrared light, and/or ultraviolet light which affects the detector by contributing noise above the read-noise of the detector.

As used herein, the term “thin film” means a film having a thickness less than 1 micron.

As used herein, the term “Tb” means terbium.

As used herein, the term “X-ray source” means an apparatus that emits X-ray radiation.

Certain example embodiments described herein may have various benefits and/or advantages to overcome the disadvantages described above. For example, certain example embodiments may improve image quality and/or the longevity of imaging devices. Certain example embodiments are portable. Certain example embodiments are easier to maintain than known X-ray devices. Thus, certain example embodiments discussed below are directed to improvements in computer-related technology, specifically, X-ray scanning and imaging technology.

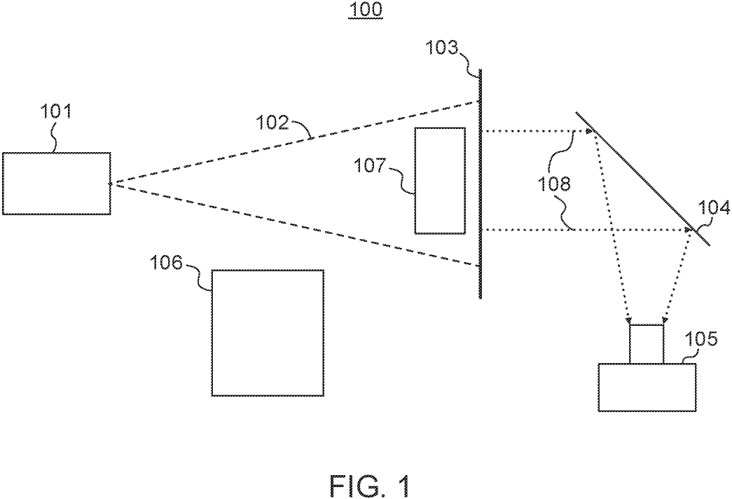

FIG. 1 illustrates an example of X-ray device 100. X-ray device 100 may include X-ray source 101 configured to emit X-rays 102 towards scintillator 103. As X-rays 102 pass through scan target 107 and collide with scintillator 103, scintillator 103 may emit visible light 108 that illuminates an image. One or more mirrors 104 may reflect visible light 108 towards detector 105.

In various example embodiments, X-ray device 100 may include motion system 106 configured to move, reposition, maneuver, or otherwise manipulate X-ray source 101, detector 105, and/or scan target 107. Furthermore, X-ray device 100 may be an X-ray CT device. In various example embodiments, X-ray source 101 may emit an X-ray cone, which may be a pencil beam, fan beam, cone beam, etc.

In some example embodiments, X-rays 102 may be emitted as a left-circular cone or right-circular cone at any angle between 20°-90°, such as 20°, 30°, 40°, 50°, 60°, 70°, 80°, or 90°. As an example, X-rays 102 emitted as a right-circular cone may include X-rays selected from soft X-rays and hard X-rays. X-rays 102 may be in the range of 20 kV - 230 kV (e.g., 20 kV, 30 kV, 40 kV, 50 kV, 60 kV, 70 kV, 80 kV, 90 kV, 100 kV, 110 kV, 120 kV, 130 kV, 140 kV, 150 kV, 160 kV, 170 kV, 180 kV, 190 kV, 200 kV, 210 kV, 220 kV, 230 kV), and/or may be in the range of 5-60 picometers (pm) (e.g., 5 pm, 10 pm, 15 pm, 20 pm, 25 pm, 30 pm, 35 pm, 40 pm, 45 pm, 50 pm, 55 pm, 60 pm). Herein, 20 kV - 225 kV may be the voltage applied to the electrode which produces X-rays 102. X-rays 102 with high photon energies above 5-10 keV (e.g., below 0.2-0.1 nm wavelength) may be referred to as “hard X-rays,” while lower photon energies (and longer wavelengths) may be referred to as “soft X-rays.”

In various example embodiments, scintillator 103 may comprise luminescent materials that illuminate when exposed to ionizing radiation, such as X-rays. Such luminescent materials may convert ionizing radiation, such as X-rays 102, into visible light 108. Scintillator 103 may comprise an organic scintillator, an inorganic scintillator, an organic-inorganic scintillator, or any combination thereof. For example, an inorganic scintillator may be selected from alkali metal halides optionally comprising dopants; phosphors; quantum dots; and combinations thereof. Additionally or alternatively, scintillator 103 may comprise at least one of gadolinium oxysulfide (Gadox), a terbium (Tb)-activated Gadox scintillator, and cesium iodide (CsI). Scintillator 103 may also comprise a thin, flexible film.

In certain example embodiments, scintillator 103 may include one or more panels of any dimension, such as 300 millimeters by 300 millimeters or 300 millimeters to 350 millimeters. Additionally or alternatively, scintillator 103 may include a plurality of mounting tabs positioned, for example, within 15, 16, 17, 18, 19, 20, 21, 22, 23, 24, or 25 millimeters of any or each edge of scintillator 103; however, mounting tabs may be located at any distance or position from the edges of scintillator 103 for mounting and/or hanging.

In some example embodiments, mirrors 104 may be fold mirrors (i.e., folded optics configured to reflect visible light 108 at a particular angle), which may be configured to reflect visible light 108 in a way to make an optical path longer than the size of a system (e.g., X-ray device 100). For example, one or more fold mirrors 104 may be configured to reflect an image of visible light 108 from scintillator 103 to detector 105, such as at a 15°, 30°, 45°, 60°, 75°, 90°, or 180° angle between scintillator 103 and detector 105. Furthermore, a first mirror of the plurality of fold mirrors may be mounted at a 15°, 30°, 45°, 60°, 75°, 90°, or 180° angle with respect to scintillator 103, and a second mirror of the plurality of fold mirrors may be mounted at a 15°, 30°, 45°, 60°, 75°, 90°, or 180° angle with respect to the first mirror. In some examples, a first fold mirror may be positioned approximately 150-350 millimeters from scintillator 103; a second fold mirror may be positioned approximately 250-350 millimeters from the first fold mirror; and detector 105 may be positioned approximately 250-350 millimeters from the second fold mirror. Alternatively, X-ray device 100 may not comprise any mirrors 104; rather, detector 105 may be positioned to detect visible light 108 emitted directly from scintillator 103 (not shown in FIG. 1).

In some example embodiments, detector 105 may comprise an optical camera, a charge-coupled device (CCD) detector, a photodiode, or any combination thereof. For example, an optical camera may comprise a complementary metal-oxide-semiconductor (CMOS) digital camera sensor. Alternatively or additionally, an optical camera may comprise a red-green-green-blue (RGGB) Bayer filter and/or a monochromatic optical camera. In other examples, an optical camera may comprise a back-side-illuminated sensor and/or front-side-illuminated sensor. As an example, detector 105 may be configured to detect infrared light, ultraviolet light, and/or visible light 108.

In some example embodiments, images from detector 105 may undergo a series of preparation and processing steps to transform detected images into usable data, including adjustments and corrections.

In some example embodiments, light collection efficiency may be improved by orienting a focal plane of detector 105 (or the reflected focal plane of mirrors 104, if mirrors 104 are used) such that a surface normal of detector 105 is parallel with a surface normal of scintillator 103, thereby minimizing the required depth of field distance of X-ray device 100. In this way, a camera lens of detector 105 may use the largest aperture possible, thereby improving light gathered and the signal-to-noise ratio of the image.

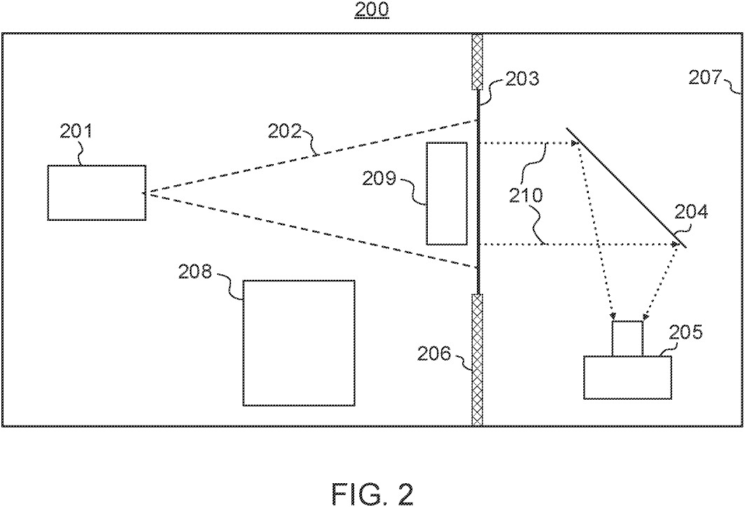

FIG. 2 illustrates an example of an X-ray device according to various example embodiments. Similar to X-ray device 100 illustrated in FIG. 1, X-ray device 200 may include X-ray source 201 configured to emit X-rays 202 towards scintillator 203. As X-rays 202 collide with scintillator 203, scintillator 203 may emit visible light 210 and project a visible image. One or more fold mirrors may reflect visible light 210 towards detector 205. Similar to FIG. 1, detector 205 may be positioned on the opposite side of scintillator 203 and shroud 206 from X-ray source 201 in order for shroud 206 to block and protect detector 205 from stray visual light and/or X-rays 202 emitted from X-ray source 201. Furthermore, a case 207 may also be positioned to completely encompass elements 201-206 and 208-210. In various example embodiments, X-ray source 201 may emit an X-ray cone, which may be a pencil beam, fan beam, cone beam, etc.

In various example embodiments, X-ray device 200 may include motion system 208 configured to move, reposition, maneuver, or otherwise manipulate X-ray source 201, detector 205, and/or scan target 209. Furthermore, X-ray device 200 may be an X-ray CT device.

In some example embodiments, X-rays 202 may be emitted as a right-circular cone comprising an angle between 20°-90°. As an example, X-rays 202 emitted as a left-circular cone or right-circular cone may include X-rays selected from soft X-rays and hard X-rays. X-rays 202 may be produced from an electrode charged to 20 kV - 225 kV, and/or may be in the range of 5 picometers - 60 picometers in wavelength.

In certain example embodiments, a length of scintillator 203 may be equal to a length of shroud 206; a height of scintillator 203 may be equal to a height of shroud 206; and/or a width of scintillator 203 may be equal to a width of shroud 206. In addition, scintillator 203 and shroud 206 may be parallel to each other (as shown) or may be at a 90° (or any other) angle to each other.

In various example embodiments, scintillator 203 may comprise an organic scintillator, an inorganic scintillator, an organic-inorganic scintillator, or any combination thereof. For example, an inorganic scintillator may be selected from alkali metal halides optionally comprising dopants, phosphors, quantum dots, and combinations thereof. Additionally or alternatively, scintillator 203 may comprise at least one of Gadox, a Tb-activated Gadox scintillator, and CsI. Scintillator 203 may also comprise a thin film.

In certain example embodiments, scintillator 203 may include a panel of any size, such as 320 millimeters by 320 millimeters. Additionally or alternatively, scintillator 203 may include a plurality of mounting tabs positioned, for example, within 20 millimeters of each edge of the panel of scintillator 203; however, mounting tabs may be mounted at any distance or position from the edges of scintillator 203 to, for example, attach to shroud 206 and/or hang from case 207 (from above, not shown).

In some example embodiments, mirrors 204 may be fold mirrors, as previously described. For example, one or more fold mirrors 204 may be configured to reflect visible light 210 from scintillator 203 to detector 205, such as at a 45°, 90°, or 180° angle between scintillator 203 and detector 205. Furthermore, a first mirror of the plurality of fold mirrors may be mounted 45° with respect to scintillator 203, and a second mirror of the plurality of fold mirrors may be mounted 90° with respect to the first mirror. In some examples, a first fold mirror may be positioned approximately 200 millimeters from scintillator 203; a second fold mirror may be positioned approximately 300 millimeters from the first fold mirror; and detector 205 may be positioned approximately 300 millimeters from the second fold mirror. Alternatively, X-ray device 200 may not include any mirrors 204; rather, detector 205 may be positioned to detect visible light 210 directly from scintillator 203.

In some example embodiments, detector 205 may comprise an optical camera, a CCD detector, a photodiode, or any combination thereof. For example, an optical camera may comprise a CMOS digital camera sensor. Alternatively or additionally, an optical camera may comprise a RGGB Bayer filter and/or a monochromatic optical camera. In other examples, an optical camera may comprise a back-side-illuminated sensor and/or front-side-illuminated sensor. As an example, detector 205 may be configured to detect infrared and/or visible light 210.

In some example embodiments, stray light may originate from X-ray source 201, an X-ray source controller (not shown), limit switches (not shown), detector 205, reflected light, light sources on the exterior of case 207, and/or any other stray light not including scintillated light emitted by scintillator 203. Rather, stray, visible light generated by elements within X-ray device 100 may be blocked by shroud 206 and/or case 207, thereby protecting detector 205 from being affected. Herein, the detector is affected when stray light produces noise above the read-noise of the detector.

Case 207 may additionally shield the detector from stray light, in addition to that which shroud 206 blocks. For example, if detector 205 detects stray, visible light, detector 205 may generate signals that include a combination of true signals from scintillator 203, as well as erroneous signals from the stray, visible light. As a result, when the signals are used in subsequent operations (e.g., reconstructing the scanned solid part), the erroneous portion of the signals may introduce errors and distortion.

Shroud 206 and/or case 207 may have an opacity of at least a minimum threshold; for example, shroud 206 and/or case 207 may block and/or absorb between between 80%-90% or 80%-99% of stray light from reaching detector 205. Herein, the percent may be a percent of the total flux of visible light photons to detector 205. In certain example embodiments, shroud 206 and/or case 207 may block stray light down to 1 ppm. In other example embodiments, shroud 206 and/or case 207 may block and/or absorb at least 80%, 90%, 95%, or 99% of stray light from reaching detector 205. Alternatively, shroud 206 and/or case 207 may block and/or absorb stray light to less than 1% of a signal, such as a signal of saturation (65535 for a 16-bit imager). In some example embodiments, the signal may refer to an image pixel intensity value. For example, when a 16-bit image pixel is completely white, the image pixel intensity value may be 65535. Similarly, when a 16-bit image pixel is completely black, the image pixel intensity value may be 0. Alternatively or additionally, shroud 206 and/or case 207 may block and/or absorb stray light at or near a read-noise of detector 205 and/or may block and/or absorb incident light on detector 205 other than scintillated light 210 from scintillator 203 to improve the quality of the image. For example, due to the intrinsic physics of an electronic imaging sensor, each pixel may include some noise. This noise may be reduced in a variety of ways, such as by cooling the sensor and/or adjusting the sensor design to isolate interfering elements. There may be a threshold amount of noise (“noise floor”) which cannot be removed. Thus, detector 205 operating in a zero-light environment may still generate a non-zero signal (i.e., pixel value). It is desirable to keep other noise sources below the noise floor so as to minimize their effect on the image.

In various example embodiments, shroud 206 and/or case 207 may be at least 50% opaque to wavelengths of light detectable by detector 205, or may be 100% opaque to wavelengths of light detectable by detector 205. Shroud 206 and/or case 207 may comprise fabric, foam, sheet metal, paper, cardboard, or any combination thereof according to the opaqueness of wavelengths of visible light detectable by detector 205.

In certain example embodiments, shroud 206 and/or case 207 may be flat and/or may have an opening allowing at least 90% of the cone of X-rays to pass through shroud 206 and/or case 207. Herein, the percent of light passing through shroud 206 may be based on brightness. Shroud 206 and/or case 207 may also include a tapered and/or truncated tip matching an optical inlet of detector 205. For example, the tip may match the optical inlet of detector 205 according to size, shape, material type, or other characteristics.

In various example embodiments, shroud 206 may be made of multiple segments, where a first segment may be longer or shorter than a second segment (as shown in FIG. 2). Furthermore, the first segment and the second segment of shroud 206 may be parallel (as shown in FIG. 2), or may form an angle less than 180°, an angle of about 40°-60°, or an angle of about 45°. Furthermore, shroud 206 and/or case 207 may be partially or completely opaque with respect to X-ray radiation, or may be partially transparent to the X-ray light cone; as an example, at least 80%, 90%, or 99% of the X-ray light cone may pass through shroud 206. Additionally or alternatively, scintillator 203 and one or more components of shroud 206 may or may not be parallel (as shown in FIG. 2).

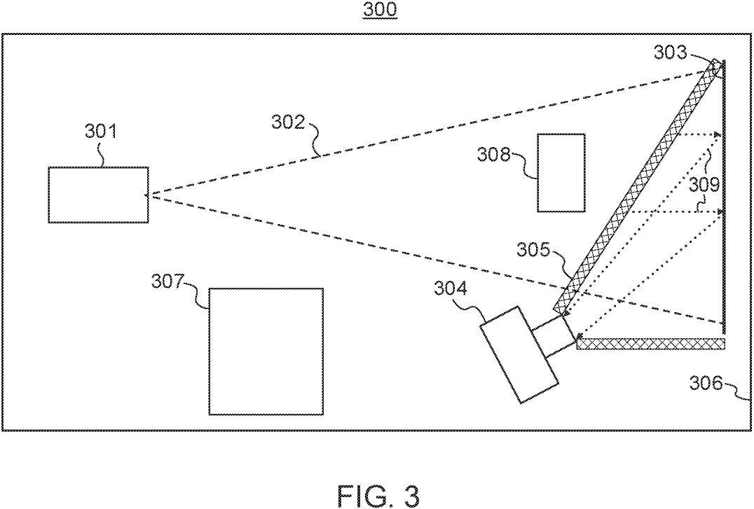

FIG. 3 illustrates an example of another X-ray device 300 according to various example embodiments, similar to X-ray devices 100 and 200 illustrated in FIGS. 1 and 2, respectively. Detector 304 (similar to detector 205) and shroud 305 (similar to shroud 206) may be positioned on the same side of scintillator 303 (similar to scintillator 203) as X-ray source 301 (similar to X-ray source 201). As a result, X-rays 302 (similar to X-rays 202) may pass through shroud 305 before contacting scintillator 303, which then transmits visible light 309 towards detector 304. In some example embodiments, shroud 305 and/or case 306 (similar to case 207) may be opaque to wavelengths of light detectable by detector 304 (i.e., infrared, visible, and ultraviolet wavelengths), but may be transparent to wavelengths of light emitted by X-ray source 301. Similar to the example embodiment of FIG. 2, the wavelengths of light emitted by X-ray source 301 may be used for scanning and/or 2-dimensional radiography (i.e., soft and hard X-rays). Without a folding mirror, X-ray device 300 may reduce losses caused by the reflection of a mirror and/or may have a more compact size. In addition, images from the initial surface of X-ray impingement may be improved, thereby avoiding blurring of the image that may be emitted by scintillator 303 as X-rays travel through the scintillating medium of scintillator 303.

In various example embodiments, X-ray device 300 may include motion system 307 configured to move, reposition, maneuver, or otherwise manipulate X-ray source 301, detector 304, and/or scan target 308. Furthermore, X-ray device 300 may be an X-ray CT device.

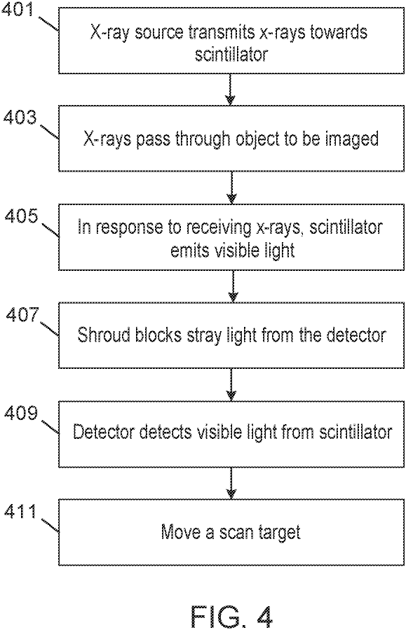

FIG. 4 illustrates an example of a flow diagram of a method that may be performed by an X-ray device, such as the X-ray devices described above, according to various example embodiments. At 401, an X-ray source may transmit X-rays, which may be in the presence of stray light, toward a scintillator, and at 403, the X-rays may pass through an object to be imaged. At 405, the scintillator may emit visible light in response to absorbing X-rays from the X-ray source. At 407, stray light may be blocked, or a portion thereof, such as by a shroud, from a detector. At 409, the detector may detect visible light from the scintillator. At 411, a motion system may move at least one of the X-ray source, the detector, and a scan target.

Certain example embodiments may be implemented in an apparatus, which may include a processor for processing information and executing instructions or operations. The processor may be any type of general or specific purpose processor. In fact, the processor may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. It should be understood that, in certain example embodiments, the apparatus may include two or more processors that may form a multiprocessor system that may support multiprocessing. In certain example embodiments, the multiprocessor system may be tightly coupled or loosely coupled (e.g., to form a computer cluster). The processor may perform functions associated with the operation of the apparatus.

The apparatus may further include or be coupled to a memory (internal or external), which may be coupled to the processor, for storing information and instructions that may be executed by the processor. The memory may be one or more memories and of any type suitable to the local application environment, and may be implemented using any suitable volatile or non-volatile data storage technology such as a semiconductor-based memory device, a magnetic memory device and system, an optical memory device and system, fixed memory, and/or removable memory. For example, the memory can be comprised of any combination of random access memory (RAM), read only memory (ROM), static storage such as a magnetic or optical disk, hard disk drive (HDD), or any other type of non-transitory machine or computer readable media. The instructions stored in the memory may include program instructions or computer program code that, when executed by the processor, enable the apparatus to perform tasks as described herein.

In an example embodiment, the apparatus may further include or be coupled to (internal or external) a drive or port that is configured to accept and read an external computer readable storage medium, such as an optical disc, USB drive, flash drive, or any other storage medium. For example, the external computer readable storage medium may store a computer program or software for execution by the processor and/or the apparatus.

In some example embodiments, the apparatus may also include or be coupled to one or more antennas for transmitting and receiving signals and/or data. The apparatus may further include or be coupled to a transceiver configured to transmit and receive information. Additionally or alternatively, in some example embodiments, the apparatus may include an input and/or output device (I/O device).

In an example embodiment, the memory may store software modules that provide functionality when executed by the processor. The modules may include, for example, an operating system that provides operating system functionality for the apparatus. The memory may also store one or more functional modules, such as an application or program, to provide additional functionality for the apparatus. The components of the apparatus may be implemented in hardware, or as any suitable combination of hardware and software.

According to some example embodiments, the processor and the memory may be included in or may form a part of processing circuitry or control circuitry. In addition, in some example embodiments, the transceiver may be included in or may form a part of transceiver circuitry.

As used herein, the term “circuitry” may refer to hardware-only circuitry implementations (e.g., analog and/or digital circuitry), combinations of hardware circuits and software, combinations of analog and/or digital hardware circuits with software/firmware, any portions of hardware processor(s) with software (including digital signal processors) that work together to cause an apparatus to perform various functions, and/or hardware circuit(s) and/or processor(s), or portions thereof, that use software for operation but where the software may not be present when it is not needed for operation. As a further example, as used herein, the term “circuitry” may also cover an implementation of merely a hardware circuit or processor (or multiple processors), or portion of a hardware circuit or processor, and its accompanying software and/or firmware. The term circuitry may also cover, for example, a baseband integrated circuit in a server, cellular network node or device, or other computing or network device.

According to certain example embodiments, the apparatus may be controlled by the memory and the processor to perform the functions associated with any of the example embodiments described herein or in the attached materials.

Although some example embodiments are described using specific examples, such as WINDOWS products and/or services, certain example embodiments described herein are not limited to these specific examples. For example, certain example embodiments described herein are applicable to any computing device and/or operating system, regardless of manufacturer, supplier, etc.

The features, structures, or characteristics of example embodiments described throughout this specification may be combined in any suitable manner in one or more example embodiments. For example, the usage of the phrases “various embodiments,” “certain embodiments,” “some embodiments,” or other similar language throughout this specification refers to the fact that a particular feature, structure, or characteristic described in connection with an example embodiment may be included in at least one example embodiment. Thus, appearances of the phrases “in various embodiments,” “in certain embodiments,” “in some embodiments,” or other similar language throughout this specification does not necessarily all refer to the same group of example embodiments, and the described features, structures, or characteristics may be combined in any suitable manner in one or more example embodiments.

Additionally, if desired, the different functions or procedures discussed above may be performed in a different order and/or concurrently with each other. Furthermore, if desired, one or more of the described functions or procedures may be optional or may be combined. As such, the description above should be considered as illustrative of the principles and teachings of certain example embodiments, and not in limitation thereof.

One having ordinary skill in the art will readily understand that the example embodiments discussed above may be practiced with procedures in a different order, and/or with hardware elements in configurations which are different than those which are disclosed. Therefore, although some example embodiments have been described based upon these example embodiments, it would be apparent to those of skill in the art that certain modifications, variations, and alternative constructions would be apparent, while remaining within the spirit and scope of the example embodiments.

We claim:

1. An X-ray device, comprising:
at least one X-ray source configured to emit an X-ray cone;
a scintillator;
a detector; and
at least one shroud positioned to block stray light from reaching the detector.

2. The X-ray device claim 1, wherein the shroud blocks at least 80% of stray light from reaching the detector.

3. The X-ray device of claim 1, wherein the shroud blocks stray light at or near a read-noise of the detector.

4. The X-ray device of claim 1, wherein the shroud blocks incident light on the detector to only scintillated light from the scintillator.

5. The X-ray device of claim 1, wherein the scintillator comprises an organic scintillator material, an inorganic scintillator material, an organic-inorganic scintillator material, or any combination thereof.

6. The X-ray device of claim 1, wherein the detector comprises an optical camera, a charge-coupled device detector, a photodiode, or any combination thereof.

7. The X-ray device of claim 6, wherein the optical camera comprises a complementary metal―oxide―semiconductor digital camera sensor.

8. The X-ray device of claim 1, wherein the shroud is positioned between the at least one X-ray source and the detector.

9. The X-ray device of claim 1, wherein the shroud is flat and has an opening allowing at least 90% of the X-ray light cone to transmit through the shroud.

10. The X-ray device of claim 1, wherein the X-ray light cone comprises a right-circular cone comprising an angle between 20°-90°.

11. The X-ray device of claim 1, further comprising one or more mirrors configured to reflect light from the scintillator to the detector.

12. The X-ray device of claim 1, wherein the detector is positioned between the shroud and the at least one X-ray source.

13. The X-ray device of claim 1, wherein the shroud is transparent to X-ray light.

14. The X-ray device of claim 1, wherein light detectable by the detector comprises infrared and visible light.

15. The X-ray device of claim 1, wherein the X-ray light cone comprises X-rays selected from soft X-rays and hard X-rays.

16. The X-ray device of claim 1, wherein the stray light is selected from light from the at least one X-ray source, light from at least one X-ray source controller, light from limit switches, light from the detector, and reflected light.

17. The X-ray device of claim 1, wherein the X-ray device is an X-ray computed tomography device.

18. The X-ray device of claim 17, further comprising:
a motion system configured to reposition the X-ray device during a scan.

19. A method, comprising:
transmitting, by at least one X-ray source and in the presence of stray light, X-rays toward a scintillator, wherein the X-rays pass through an object to be imaged;
emitting, by a scintillator, visible light in response to receiving the X-rays;
blocking the stray light, or a portion thereof, from the detector by a shroud; and
detecting, by a detector, the visible light from the scintillator.

20. The method of claim 19, further comprising:
moving, by a motion system, at least one of the at least one X-ray source, the detector, and a scan target.
